(19) United States
(12) Patent Application Publication
(10) Pub. No.: US 2015/0071935 A1
(43) Pub. Date: Mar. 12, 2015

(54) Treatment for the rapid amelioration of clinical depression

(71) Applicant: Marv Enterprises, LLC, Hermitage, PA (US)

(72) Inventor: Mitchell S. Felder, Hermitage, PA (US)

(21) Appl. No.: 14/390,199

(22) PCT Filed: May 21, 2013
(86) PCT No.: PCT/US2013/041931

§ 371 Date: Oct. 2, 2014

Related U.S. Application Data
(60) Provisional application No. 61/652,231, filed on May 27, 2012.

(51) Int. Cl. A61K 35/30

(52) U.S. Cl. CPC A61K 35/30; A61K 2035/122
USPC 424/140.1

(57) ABSTRACT

The present invention relates to an article and method of extracorporeal treating a patient's body fluid, for example, CSF (cerebrospinal fluid), lymph, or blood. The treatment includes a plurality of stages comprising removing the body fluid from a patient, applying an extracorporeal treatment to the body fluid, and returning the body fluid to the patient. In the first stage of the treatment, the body fluid is removed from the patient. A convenient method for removing blood is utilizing standard venipuncture technique. A convenient method for removing CSF is using a standard lumbar puncture. In the second stage, a treatment is applied to the body fluid. The treatment can include an antibody directed against targeted antigen(s)/TA(s). The third stage comprises returning the body fluid to the patient, and can also include removing the treatment from the body fluid.

### CROSS-REFERENCE TO RELATED APPLICATIONS

This application claims benefit under 35 U.S.C. §119(e) of U.S. Patent Application No. 61/652,231, filed May 27, 2012, which is hereby incorporated herein by reference in its entirety.

### FIELD OF THE INVENTION

The present invention relates to a treatment for clinical depression using an extracorporeal methodology to achieve this purpose.

### BACKGROUND OF THE INVENTION

Clinical depression is a mental disorder characterized by low self-esteem and accompanied by low mood, and by loss of pleasure or interest in normally enjoyable activities. Major depressive disorder is a disabling condition that adversely affects a person's family, work or school life, sleeping and eating habits, and general health. In the United States, around 3.4% of people with major depression commit suicide, and approximately 60% of people who commit suicide had depression or another mood disorder. Up to thirty percent of primary care patients have been shown to have clinical depression.

The diagnosis of major depressive disorder is based on the patient's self-reported experiences, behavior reported by relatives and friends, and a standard mental status examination. There is no specific laboratory test for major depression, although physicians generally request tests for those physical conditions that may cause similar symptoms. The most common time of onset is between the ages of 20 and 30 years, with a later peak between 30 and 40 years. Standardized screening tools such as Major Depression Inventory can be used to detect major depressive disorder. Patients with clinical depression are oftentimes treated with antidepressant medication. In many cases, patients concomitantly receive psychotherapy.

Hospitalization is necessary in cases in which a patient has significant risk of harm to self or others. Depressed individuals have shorter life expectancies than those without depression, in part because of greater susceptibility to medical illnesses and suicide. Clinical depression is a major contributor to almost all known diseases. The alleviation of clinical depression would therefore be extremely useful in decreasing morbidity and mortality in this patient group.

### SUMMARY OF THE INVENTION

The present invention relates to an article and method of extracorporeal treating a patient's body fluid, for example, CSF (cerebrospinal fluid), lymph, or blood. U.S. Ser. No. 13/128,870, U.S. Ser. No. 13/128 177, U.S. Ser. No. 13/254,855, U.S. 61/612,474, and U.S. 61/644,292 are hereby incorporated by reference.

The treatment includes a plurality of stages comprising removing the body fluid from a patient, applying an extracorporeal treatment to the body fluid, and returning the body fluid to the patient.

In the first stage of the treatment, the body fluid is removed from the patient. A convenient method for removing blood is utilizing standard venipuncture technique. A convenient method for removing CSF is utilizing a standard lumbar puncture. In the second stage, a treatment is applied to the body fluid. The treatment can include an antibody directed against targeted antigen(s)/TA(s). The third stage comprises returning the body fluid to the patient, and can also include removing the treatment from the body fluid.

### BRIEF DESCRIPTION OF THE DRAWING

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

The method of the present invention comprises treating a patient's body fluid extracorporeally with antibody(s) designed to react with particular targeted antigen(s)/TA(s): TNF-alpha (Tumor Necrosis Factor-alpha) and Interleukin-6(IL-6). The antibody can include a moiety, for example, an albumin moiety, that can complex with the target antigen/TA and thereby permit efficacious dialysis of the antibody-antigen complex. Dialysis methods are well known by one skilled in the art.

In an embodiment of the invention, the antibody comprises an albumin moiety and targets the removal of the TA from the body fluid.

The target antigen(s)/TA(s): TNF-alpha (Tumor Necrosis Factor-alpha) and Interleukin-6 (IL-6) can be differentiated utilizing standard ELISA methodology. ELISA (enzyme-linked immunosorbant assay) is a biochemical technique which allows for the detection of an antigen in a sample. In ELISA an antigen is affixed to a surface, and then an antibody is utilized for binding to the antigen. The antibody is linked to an enzyme which enables a color change in the substrate.

Other strategies may be employed to validate the level of target antigen(s)/TA(s) in the body fluid: Western blotting technology, UV/Vis spectroscopy, mass spectrometry, and surface plasmon resonance (SPR).

An alternative methodology of the present intervention would utilize a designer antibody with an attached macromolecular moiety instead of an albumin moiety. The macromolecular moiety, attached to the antibody, would be 1.000 mm to 0.00001 mm in diameter. The antibody-macromolecular moiety-targeted antigen complex would then be blocked from reentering the patient's body fluid, by utilizing a series of microscreens which contain openings with a diameter 50% to 99.99999% less than the diameter of the designer antibody-macromolecular moiety. The microscreen opening(s) must have a diameter of at least 25 micrometers in order to allow for the passage and return to circulation of the nonpathologic inducing body fluid constituents.

Alternatively, the target antigen(s)/TA(s): TNF-alpha (Tumor Necrosis Factor-alpha) and Interleukin-6, may be captured by utilizing antibody microarrays which contain antibodies to the target antigen(s). The antibody microarrays are composed of millions of identical monoclonal antibodies attached at high density on glass or plastic slides. After sufficient extracorporeal exposure of the TA(s) to the antibody microarrays, the antibody microarrays-TA(s) may be disposed of, using standard medical practice.

Another alternative methodology of the present intervention comprises removing the targeted antigen(s)/TA(s): TNF-alpha (tumor necrosis factor-alpha) and interleukin-6 (IL-6), from the body fluid using a designer antibody containing an iron (Fe) moiety. This will then create an Fe-antibody-antigen complex. This iron-containing complex may then be efficaciously removed utilizing a strong, localized magnetic force field.

Alternatively, immunoaffinity chromatography may be employed in which the heterogeneous group of molecules in the body fluid will undergo a purification process. There will be an entrapment on a solid or stationary phase or medium. Only the targeted antigens (TAs) will be trapped utilizing immunoaffinity chromatography. A solid medium can be removed from the mixture, washed, and the TA(s) may then be released from the entrapment through elution.

Alternatively, gel filtration chromatography may be utilized in which the body fluid is used to transport the sample through a size exclusion column that will be used to separate the target antigen(s)/TA(s) by size and molecular weight.

Another alternative methodology of the present intervention would utilize a molecular weight cut-off filtration. Molecular weight cut off filtration refers to the molecular weight at which at least 80% of the target antigen(s)/TA(s) is prohibited from membrane diffusion.

The invention comprises at least three stages including a first stage, a second stage and a third stage. The first stage comprises removing body fluid from a patient. The second stage treats the body fluid. The third stage returns the treated body fluid to the patient after having achieved the physical removal of the targeted antigen(s)/TA(s): TNF-alpha (tumor necrosis factor-alpha) and interleukin-6 (IL-6).

The treatment can include the removal of the targeted antigen(s). The cleansed body fluid can then be returned to the patient, such as, for example by using the same catheter that was originally used in removing the body fluid. In one embodiment, the treatment of body fluid comprises removing 25 ml to 500 ml of body fluid from a patient, and then applying the treatment to the body fluid before returning it to the patient. The frequency of such treatments would depend upon an analysis of the underlying symptomatology and pathology of the patient.

The article of the invention includes two-stages. The first stage includes an inlet for body fluid and at least one exterior wall defining a treatment chamber that is fluidly connected to a second stage. The second stage comprises a removal module and an outlet for the body fluid. In embodiments, the removal module is selected from a group comprising a mechanical filter, a chemical filter, a dialysis machine, a molecular filter, molecular adsorbant recirculating system (MARS), a plasmapharesis unit, or combinations thereof.

The method includes removing body fluid from a patient in a first stage, treating the body fluid to obtain a reduction in the target antigen(s), and optionally removing the treatment from the body fluid in a second stage, and returning the body fluid to the patient in a third stage. The body fluid can be removed from the patient using any convenient method, including standard venipuncture procedure. The body fluid can be removed from the patient using any convenient method, including a standard lumbar puncture. The second stage can include sequentially passing the extracorporeal bodily fluid through a treatment chamber and a removal module.

The second stage applies a treatment to the body fluid, which can include introducing a designer antibody that joins with a targeted antigen (TA) in the body fluid to form an antibody-antigen complex. The antibody-antigen complex can be removed from the body fluid in the removal module. Optionally, the antibody-antigen complex can be conjugated with a second antibody comprising a moiety that increases the efficacy of removal to form an antibody-moiety-antigen complex.

In the third stage, the purified body fluid (body fluid with removed TA(s): TNF-alpha (tumor necrosis factor-alpha) and interleukin-6 (IL-6), is then returned to the patient.

The device of the invention comprises a first stage including an inlet for body fluid and at least one exterior wall defining a treatment chamber that is fluidly connected to a second stage comprising a removal module and an outlet for the body fluid to be treated. The treatment chamber can include a delivery tube for introducing a treatment into the treatment chamber. In embodiments, the delivery tube comprises a hollow tube including at least one interior wall defining a plurality of holes through which the treatment can be added to the treatment chamber. The treatment can also be delivered through the hollow tube in counter-current mode with reference to the flow of the extracorporeal body fluid. The removal module can be any device capable of removing the antibody-antigen complex. In embodiments, the removal module is selected from a group comprising a mechanical filter, a chemical filter, a dialysis machine, a molecular filter, molecular adsorbant recirculating system (MARS), a plasmapharesis unit, or combinations thereof.

In an example, the first stage of the device applies a treatment of an antibody with an attached albumin moiety that targets the antigen(s): TNF-alpha (tumor necrosis factor-alpha) and interleukin-6 (IL-6). The second stage includes substantial removal of the treatment from the extracorporeal bodily fluid.

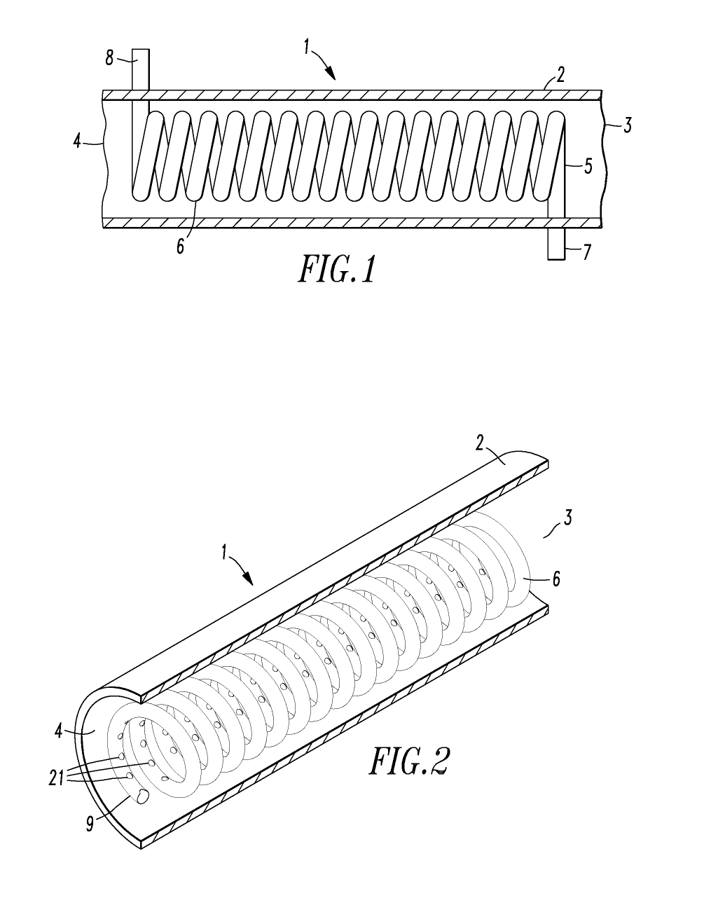

As shown in FIG. 1, the first stage can include an exterior wall 2 defining a treatment chamber 5. The treatment can be applied in the treatment chamber 5. Residence times of the body fluid to be treated can be altered by changing the dimensions of the treatment chamber or the flow rate of the body fluid through the treatment chamber 5. The body fluid to be treated enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the body fluid. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer antibodies in the body fluid. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic.

The antibody with attached albumin moiety, targeting the antigen/TA(s): TNF-alpha (tumor necrosis factor-alpha) and interleukin-6 (IL-6) can be delivered in a concurrent or counter-current mode with reference to the body fluid. In counter-current mode, the body fluid enters the treatment chamber 5 at the inlet 3. The designer antibody can enter through a first lead 8 near the outlet 4 of the treatment chamber 5. Body fluid then passes to the outlet 4 and the designer antibodies pass to the second lead 7 near the inlet 3. The removal module of the second stage substantially removes the designer antibodies-antigen molecular compound from the body fluid.

The second stage can include a filter, such as a dialysis machine, which is known to one skilled in the art. The second stage can include a molecular filter. For example, molecular adsorbants recirculating system (MARS), which may be compatible and/or synergistic with dialysis equipment. MARS technology can be used to remove small to average sized molecules from the body fluid. Artificial liver filtration presently uses this technique.

The methodology can include a plurality of steps for removing the targeted antigen(s)/TA(s): TNF-alpha (tumor necrosis factor-alpha) and interleukin-6 (IL-6). A first step can include directing a first antibody against the targeted antigen. A second step can include a second antibody. The second antibody can be conjugated with albumin, or alternatively a moiety which allows for efficacious dialysis. The second antibody or antibody-albumen complex combines with the first antibody forming an antibody-antibody-moiety complex. A third step is then utilized to remove the complex from the body fluid. This removal is enabled by utilizing dialysis and/or MARS. The purified body fluid can then be returned to the patient.

In practice, a portion of the purified body fluid can be tested to ensure a sufficient portion of the targeted antigen: TNF-alpha (tumor necrosis factor-alpha) and interleukin-6 (IL-6), has been successfully removed from the body fluid. Testing can determine the length of treatment and evaluate the efficacy of the sequential dialysis methodology in removing the targeted antigens. The body fluid with an unacceptably large concentrations of complex remaining can then be refiltered before returning the body fluid to the patient.

In embodiments, the second stage to remove the antibody-moiety-targeted antigen complex by various techniques including, for example, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with attached albumin moiety targeting the antigen(s): TNF-alpha (tumor necrosis factor-alpha) and interleukin-6 (IL-6).

Additional embodiments can include continuous renal replacement therapy (CRRT) which can remove large quantities of filterable molecules from the extracorporeal body fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) is the ratio of the molecular concentration in the filtrate to the incoming bodily fluid. A SC close to zero implies that the moiety antibody-targeted antigen complex will not pass through the filter. A filtration rate of 10 ml per minute is generally satisfactory. Other methods of increasing the removability of the moiety-antibody-targeted antigen include the use of temporary acidification of the bodily fluid utilizing organic acids to compete with protein binding sites.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for treating body fluid, comprising:
a. removing body fluid from a patient;
b. applying a treatment to the body fluid; and
c. returning the body fluid to the patient.

2. The method of claim 1, further comprising removing the treatment from the body fluid before returning the body fluid to the patient.

3. The method of claim 1, wherein the treatment includes:
a. introducing an antibody that joins with an antigen in the body fluid to form an antibody-antigen complex; and
b. removing the complex from the body fluid.

4. The method of claim 3, wherein the targeted antigen (TA) is selected from a group consisting of TNF-alpha (Tumor Necrosis Factor-alpha), Interleukin-6 (IL-6) and combinations thereof.

5. The method of claim 1, wherein the treatment includes:
a. introducing an antibody that joins with a targeted antigen in the body fluid to form an antibody-antigen complex; and
b. conjugating the antibody-antigen complex with a second antibody comprising a moiety that increases the efficacy of removal to form an antibody-moiety-antigen complex.

6. The method of claim 2, further comprising testing the body fluid after the treatment and before returning the body fluid to the patient in order to determine efficacy of treatment.
